(19) United States
(12) Patent Application Publication
(10) Pub. No.: US 2014/0194368 A1
(43) Pub. Date: Jul. 10, 2014

(54) Method of Treating Cancer by Administration of Low Levels of Heat Shock Protein 70 (HSP70)

(71) Applicant: BEECH TREE LABS, INC., Delanson, NY (US)

(72) Inventor: John McMichael, Delanson, NY (US)

(73) Assignee: Beech Tree Labs, Inc., Delanson, NY (US)

(21) Appl. No.: 13/733,946

(22) Filed: Jan. 4, 2013

(51) Int. Cl. A61K 38/17

(52) U.S. Cl. CPC A61K 38/1709
USPC 514/19.4; 514/19.3; 514/19.5

(57) ABSTRACT

The invention is directed to methods of treating cancer by administration of heat shock protein 70 (Hsp70) to the subject suffering from cancer.

### CROSS-REFERENCE TO RELATED APPLICATION

This application claims the benefit of priority to U.S. Provisional Application No. 61/583,535, filed Jan. 5, 2012, the disclosure of which is incorporated herein by reference in its entirety.

### BACKGROUND OF THE INVENTION

Heat shock proteins are a class of functionally related proteins involved in the folding and unfolding of other proteins. There are several different heat shock proteins which are named according to their weights. Heat shock proteins are expressed when cells are exposed to elevated temperatures or other stresses such as infection, inflammation, exposure to toxins, starvation and water deprivation, hypoxia, radiation exposure and the like.

There exist a variety of heat shock proteins which are named according to their approximate molecular weights. Hsp 60, Hsp 70, Hsp90 and Hsp 100 are different proteins where the number reflects their approximate molecular weight in kilodaltons. The major heat shock proteins are expressed at high levels in stressed cells but occur at low to moderate levels in cells which have not been stressed. As one example, Hsp70 is hardly detectable at normal temperatures but is highly expressed in cells upon heat shock. In contrast, Hsp60 and Hsp90 are more highly expressed under normal conditions but have their expression further induced by heat.

Heat Shock Protein 70 (Hsp70) is a chaperone protein with an approximate weight of 70 kilodaltons that acts as a quality control mechanism to protect amino acids as they align to become a protein. It appears to be up-regulated in transformed cells, consistent with the tendency of those cells to exhibit an increased metabolic rate compared to normal cells. Because of their apparent relationship to cancer there is much interest in heat shock proteins as a target for cancer prevention and treatment.

In particular, some workers believe that heat shock proteins might be involved in binding protein fragments from dead malignant cells and then presenting them to the immune system. Heat shock proteins are used as immunologic adjuvants in boosting responses to vaccine.

Inhibitors of heat shock proteins are also thought to be promising as anti-cancer therapeutics because such heat shock proteins are overexpressed in cancer cells and thought to be important to the survival of those cells. See, Didelot, et al. “Anti-cancer therapeutic approaches based on intracellular and extracellular heat shock proteins”. Curr. Med. Chem. 14 (27): 2839-47 (2007). See also Solit, et al. “Hsp90: a novel target for cancer therapy”. Curr Top Med Chem 6 (11): 1205-14 (2006).

Multhoff, U.S. Pat. No. 7,700,737 discloses anti-Hsp70 antibodies for use in diagnosis and therapy of primary and metastatic neoplastic diseases. A potential limitation of such therapy is that treating a cancer patient with anti-Hsp70 monoclonal antibodies may well destroy the cancer cell target, but would simultaneously damage healthy cells for which Hsp70 activity is essential.

Of interest to the application is the disclosure of Srivastava U.S. Pat. No. 6,984,389 which discloses the administration of heat shock proteins including Hsp60, Hsp70, Hsp90, Hsp110, gp96 or caireticulin in conjunction with a non-vaccine treatment modality such as a tyrosine kinase inhibitor (imatinib mesylate, Gleevec™) for the treatment of cancer or infectious diseases. In particular the Hsp preparation is said to comprise “HSP-peptide complexes which display the antigenicity of an agent of the infectious disease.” (col. 8, lines 31-33). Dosages of HSP's are said to range from 0.1 μg to 1000 μg per administration with preferred dosages of Hsp70 said to be in the range of from 10 to 600 μg per administration if administered intradermally. (col. 50, lines 36-49)

Also of interest is the disclosure of Srivastava U.S. Pat. No. 7,666,581 which is directed to methods of treating infectious diseases and cancer by complexing antigenic proteins or peptides to heat shock proteins.

In addition, Srivastava U.S. Pat. No. 8,029,808 discloses the administration of heat shock proteins combination with other anticancer therapeutics including complexes of heat shock proteins with such anticancer therapeutics. Dosages of HSP's are said to range from 0.1 μg to 1000 μg per administration with preferred dosages of Hsp70 said to be in the range of from 10 to 600 μg per administration if administered intradermally.

Accordingly, there remains an interest in alternative therapies which might decrease Hsp70 production in cancer cells and conceivably induce the selective demise of that population.

### BRIEF SUMMARY OF THE INVENTION

The present invention relates to the discovery that administration of small dosages of HSP70 is useful in the treatment of various cancers. While not wishing to be bound by any particular theory of the invention it is believed that the introduction of low levels of HSP70 may induce a negative feedback loop in transformed cells such that the endogenous production of HSP70 in those cells would be decreased. Such a reduction would render the cancer cells more susceptible to attacks by the subject's own immune system and other cancer therapies.

Specifically, the invention provides a method of treating cancer in a subject in need thereof comprising: administering an effective amount of heat shock protein 70 (Hsp70) to said subject. The subject can be any mammal including a human and the cancer is selected from the group of cancers comprising brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), sarcomas (including osteosarcoma, chondrosarcoma and fibrosarcoma).

The Hsp70 is preferably administered in the absence of any vaccine and more preferably is not complexed to any other molecule.

The method is particularly useful because the Hsp70 is administered at a relatively low dosages. According to one aspect of the invention the Hsp70 can be administered at daily dosages of from 0.0003 micrograms to 3 micrograms per day, with daily dosages of from 0.003 micrograms to 0.3 micrograms per day being particularly preferred. The dosage is preferably administered in one or more doses during the day with four dosages totaling about 0.03 micrograms per day being particularly preferred.

The Hsp70 may be administered by a variety of modes including a mode selected from the group consisting of sublingual, bucal, oral drench, subcutaneous, intramuscular, intradermal, or intravenous administration although sublingual administration is particularly preferred.

### BRIEF DESCRIPTION OF THE DRAWINGS

FIG. 1 depicts transcription levels of Hsp70 mRNA in cancerous cell lines treated with Hsp70 protein.

### DETAILED DESCRIPTION OF THE INVENTION

The present invention relates to the discovery that administration of small dosages of HSP70 is useful in the treatment of various cancers. In some embodiments, the cancer is selected from the group of cancers comprising brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), sarcomas (including osteosarcoma, chondrosarcoma and fibrosarcoma).

It has been found that in treatment of humans and non-human animals with a variety of malignancies that no adverse effects were identified and further that dogs and cats live longer with HSP70 treatment than do historical controls. Further, human patients experienced reduced cancer-associated pain, and showed improved stamina and vitality. The treatment also appeared to decrease the rate of disease progression in the human subjects.

### Example 1

According to this example two breast adenocarcinoma cell lines (MDA MB 231) and (MDA MB 231 T) cells were grown in DMEM 10% FBS and treated with various dosages of Hsp70 protein (Novus Fine Chemicals) for 24 hours. One drop of a solution comprising 0.0068 micrograms of Hsp70 in PBS saline was added to each well in a standard 96 well cell culture plate. In addition, identical experiments were run at 0.1× and 10× concentrations of Hsp70.

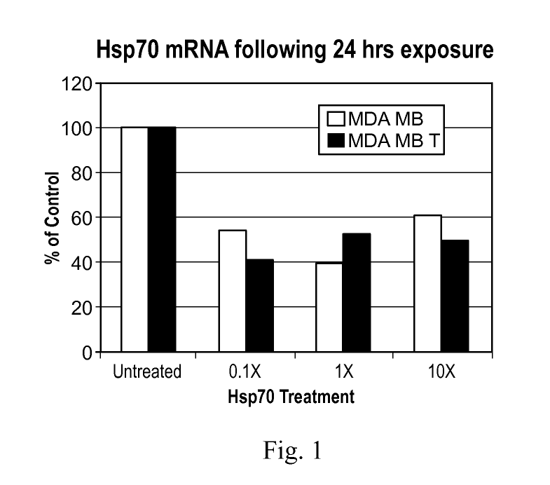

Following 24 hours of exposure cells were collected to determine endogenous production of Hsp 70 RNA. The cells were subjected to RNA extraction by Trizol and RNA quality and quantity were measured by a Nano-drop spectrophotometer (Thermo-Fisher Scientific, Wilmington, Del.). RT-PCR (Real-time PCR) and qPCR were conducted according to the manufacturer's protocol (Quigen) to determine levels of Hsp70 mRNA. The results presented in FIG. 1 show that treatment with Hsp70 reduced endogenous Hsp70 transcription by 40-60% in the treated breast cancer cells. Without being bound by a theory of the invention it is believed that the presence of the exogenous Hsp 70 functions to downregulate the production of endogenous Hsp 70 by the tumor. Because the Hsp 70 is a required protein for the tumor cells, remission is thereby induced.

### Example 2

According to this example, a 33 year old male was diagnosed with a stage 4 glioblastoma multiform following a seizure and was treated in accordance with the invention. After the subject's original diagnosis, sequential MRIs showed a rapidly progressing tumor. Two rounds of surgery and one round each of chemotherapy and radiation failed to eliminate the cancer. Eight months after the original diagnosis, the patient was admitted to the hospital in a rapidly deteriorating progressing over a 3-4 hour period during which he suffered loss of motor skills, speech, and finally consciousness. He was labeled “comatose” upon admission. Following aggressive intervention of several types, he was discharged four days later in a weakened state with impaired cognition and both gross and fine motor function.

Within two days of discharge, he was treated by four times daily sublingual administration of one drop of a solution of Hsp70 at a per drop concentration of 0.0068 micrograms in PBS saline. No other changes were made to the subject's therapeutic regimen except for the discontinuation of an anti-tumor agent (Avastin).

Notable progress was experienced beginning shortly after the initiation of Hsp70 therapy. Cognition, speech, and energy improvements were followed by improved fine motor control (able to tie shoes and insert contact lenses for first time in months), balance, and endurance. At five weeks, he could mow the lawn, clean house, and walk unassisted for more than a mile. An MRI taken two months post-discharge showed no change in tumor size.

### Example 3

According to this example, peripheral blood mononuclear cells (PBMCs) were treated with various doses of Hsp70 (0.1×, 1× or 10×) for 24 hours. Following total mRNA extraction, Cancer Pathway Finder qPCR arrays were utilized to determine gene expression of the PBMCs compared to untreated controls. Results indicated that PBMCs treated with Hsp70 demonstrated downregulated expression of ARNT and the urokinase plasminogen activator, SERPINB2, compared to the untreated controls. It is known in the art that expression of ARNT and/or SERPINB2 is associated with tumor development and invasiveness. Thus, this data confirms the that Hsp70 is useful in treating cancer a subject by downregulating the expression of at least two tumor-expressing genes (e.g., ARNT and SERPINB2).

It is anticipated that numerous variations and modification of the embodiments of the invention described above will occur to those of ordinary skill in the art when apprised of the teachings of the present specification. Accordingly, only such limitations as appear in the appended claims should be placed thereon.

What is claimed is:

1. A method of treating cancer in a subject in need thereof comprising:
administering an effective amount of heat shock protein 70 (Hsp70) to said subject.

2. The method of claim 1 wherein the subject is human.

3. The method of claim 1 wherein the cancer is selected from the group of cancers comprising brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), sarcomas (including osteosarcoma, chondrosarcoma and fibrosarcoma).

4. The method of claim 1 wherein the cancer is a brain tumor.

5. The method of claim 1 wherein the Hsp70 is administered at a dosage of from 0.0003 micrograms to 3 micrograms per day.

6. The method of claim 1 wherein the Hsp70 is administered at a dosage of from 0.003 micrograms to 0.3 micrograms per day.

7. The method of claim 1 wherein the Hsp70 is administered by is administered by a mode selected from the group consisting of sublingual, bucal, oral drench, subcutaneous, intramuscular, intradermal, or intravenous.

8. The method of claim 1 wherein the Hsp70 is administered sublingually.

9. The method of claim 1 wherein the Hsp70 is administered in the absence of other cancer therapeutics.

10. The method of claim 1 wherein the Hsp70 is administered in the absence of any vaccine.

11. The method of claim 1 wherein the Hsp70 is not complexed to any other molecule.
